SEQUENCE LISTING (20 sequences)

SEQ ID NO: 1 - DNA, 247 bp, Artificial
  targeting element
gggacgacgatgacacgatactttgtcggccgaactcgctgctccgatccggcgagatcgcagggtgttgctattcgcgtgccgtgtgcatacgccgatcacatgaccagggacgacgatgacacgatactttgtcggccgaactcgctgtttaactgcccggcgagatcgcagggtgttgtgctattcgcgtgccgtgtgcatacgccgatcacatgaccaaccctgccgcctggactccgcctgt

SEQ ID NO: 2 - DNA, 44 bp, Artificial
  Purification element
accgaccagaatcatgcaagtgcgtaagatagtcgcgggccggg

SEQ ID NO: 3 - RNA, 12 nt, Artificial
  Targeting element
accaggcuugga

SEQ ID NO: 4 - RNA, 8 nt, Artificial
  Targeting element
ucacaugg

SEQ ID NO: 5 - RNA, 12 nt, Artificial
  Targeting element
cuuggaagcaga

SEQ ID NO: 6 - RNA, 12 nt, Artificial
  Targeting element
ucuucucggauu

SEQ ID NO: 7 - RNA, 12 nt, Artificial
  Targeting element
ggccagggguuc

SEQ ID NO: 8 - RNA, 10 nt, Artificial
  Targeting element
aggaacgaac

SEQ ID NO: 9 - RNA, 12 nt, Artificial
  Targeting element
uauguggccauc

SEQ ID NO: 10 - RNA, 12 nt, Artificial
  Targeting element
accaggcuugga

SEQ ID NO: 11 - RNA, 10 nt, Artificial
  Targeting element
cagcgagacc

SEQ ID NO: 12 - RNA, 12 nt, Artificial
  Targeting element
cucacuugggag

SEQ ID NO: 13 - RNA, 10 nt, Artificial
  Targeting element
agcaccaccu

SEQ ID NO: 14 - RNA, 12 nt, Artificial
  Targeting element
caggagucuaca

SEQ ID NO: 15 - RNA, 12 nt, Artificial
  Targeting element
ggagaagaaggc

SEQ ID NO: 16 - RNA, 12 nt, Artificial
  Targeting element
ggggaaccugca

SEQ ID NO: 17 - RNA, 10 nt, Artificial
  Targeting element
accaaugggg

SEQ ID NO: 18 - RNA, 12 nt, Artificial
  Targeting element
ggggaaccugca

SEQ ID NO: 19 - DNA, 101 bp, Artificial
  Triple helix motif
aaaaaaaaaaaaaaaaaaaaaaaaatatgtgaccctatttttggaacggcagaccagtgacctccacaggccacgcttgttcctttttgggtcggttttgt

SEQ ID NO: 20 - DNA, 101 bp, Artificial
  Triple helix motif
tgttttggctgggtttttccttgttcgcaccggacacctccagtgaccagacggcaaggtttttatcccagtgtataaaaaaaaaaaaaaaaaaaaaaaaa

(19) United States
(12) Patent Application Publication
(10) Pub. No.: US 2021/0277393 A1
(43) Pub. Date: Sep. 9, 2021

(54) CIRCULARIZED ENGINEERED RNA AND METHODS

(71) Applicants: Christopher Livia, Rochester, MN (US); Atta Behfar, Rochester, MN (US); Michael Sabbah, Rochester, MN (US); MAYO FOUNDATION FOR MEDICAL EDUCATION AND RESEARCH, Rochester, MN (US)

(72) Inventors: Christopher Livia, Rochester, MN (US); Atta Behfar, Rochester, MN (US); Michael Sabbah, Rochester, MN (US)

(21) Appl. No.: 17/261,825

(22) PCT Filed: Jul. 24, 2019
(86) PCT No.: PCT/US19/43174

§ 371 Date: Jan. 20, 2021

Related U.S. Application Data
(60) Provisional application No. 62/702,853, filed on Jul. 24, 2018.

(51) Int. Cl. C12N 15/11; C12N 15/86; C12N 15/10

(52) U.S. Cl. CPC C12N 15/111; C12N 15/86; C12N 15/1003; C12N 2840/206; C12N 2830/48; C12N 2840/44; C12N 2840/60; C12N 2310/16; C12N 2310/15; C12N 2310/532

(57) ABSTRACT

A circular RNA molecule generally includes at least one coding region and an internal ribosome entry site (IRES) operably linked to the coding region. The RNA may be more resistant to digestion by an RNA endonuclease that a linear form of the circular RNA. In another aspect, a polynucleotide generally includes a transcription unit and a promoter operably linked to the transcription unit. The transcription unit includes a circularizing element, at least one coding region and an internal ribosome entry site (IRES) operably linked to the coding region. When transcribed by a cell, the transcribed RNA forms a circular RNA molecule.

### CROSS-REFERENCE TO RELATED APPLICATION

This application claims the benefit of U.S. Provisional Patent Application No. 62/702,853, filed Jul. 24, 2018, which is incorporated herein by reference in its entirety.

### SUMMARY

This disclosure describes, in one aspect, a polynucleotide comprising that includes a transcription unit and a promoter operably linked to the transcription unit. The transcription unit includes a circularizing element, at least one coding region and an internal ribosome entry site (IRES) operably linked to the coding region. The circularizing element includes a first sequence at the 5′ end of the transcription unit and a second sequence at the 3′ end of the transcription unit. The coding region is located between first sequence of the circularizing element and the second sequence of the circularizing element.

In various embodiments, the polynucleotide can be DNA or RNA.

In some embodiments, the first sequence of the circularizing element includes a first portion of intron from thymidylate synthetase (td) of bacteriophage RNA and the second sequence of the circularizing element includes a second portion of the intron from thymidylate synthetase (td) of bacteriophage RNA. In other embodiments, the first sequence of the circularizing element includes a eukaryotic splice acceptor sequence and the second sequence of the circularizing element includes a eukaryotic splice donor sequence.

In some embodiments, the IRES comprises cricket paralysis virus IRES (CrPV-IRES) or Plautia stali intestine virus IRES (PSIV-IRES).

In some embodiments, the coding region encodes a therapeutic peptide.

In some embodiments, the IRES is operably linked to at least two coding regions.

In some embodiments, the polynucleotide further includes a second IRES operably linked to a second coding region.

In some embodiments, the transcription unit further includes one or more of the following: a triple helix motif, an untranslated region (UTR), an RNA stability element, an RNA export element, or an affinity purification aptamer.

In another aspect, this disclosure describes a circular RNA molecule that generally includes at least one coding region and an internal ribosome entry site (IRES) operably linked to the coding region.

In some embodiments, the IRES comprises cricket paralysis virus IRES (CrPV-IRES) or Plautia stali intestine virus IRES (PSIV-IRES).

In some embodiments, the coding region encodes a therapeutic peptide.

In some embodiments, the IRES is operably linked to at least two coding regions.

In some embodiments, the circular RNA molecule further includes a second IRES operably linked to a second coding region.

In some embodiments, the circular RNA molecule further includes one or more of the following: a triple helix motif, an untranslated region (UTR), an RNA stability element, an RNA export element, or an affinity purification aptamer.

In some embodiments, the circular RNA is more resistant to digestion by an RNA endonuclease than a linear form of the circular RNA.

In another aspect, this disclosure describes a cell transformed with any embodiment of the polynucleotide summarized above.

In another aspect, this disclosure describes a cell that includes any embodiment of the circular RNA molecule summarized above.

In another aspect, this disclosure describes a method of making a circular RNA molecule. Generally, the method includes transforming a host cell with any embodiment of the polynucleotide summarized above, allowing the host cell to transcribe a linear RNA molecule from the polynucleotide, and allowing the circularizing element to circularize the linear RNA molecule, thereby forming a circular RNA molecule.

In some embodiments, the method further includes isolating at least a portion of the circular RNA molecules from the host cell.

In some embodiments, the method further includes digesting linear RNA molecules with an RNase and collecting undigested circular RNA molecules.

In some embodiments, the circular RNA molecule includes an extracellular vesicle targeting sequence effective for transferring the circular RNA to an exosome in the host cell. In some of these embodiments, the method further includes isolating exosome containing the circular RNA molecules.

In another aspect, this disclosure describes a method of treating a subject in need of therapy provided by a therapeutic peptide. Generally, the method includes administering to the subject either any embodiment of the polynucleotide summarized above or any embodiment of the circular RNA molecule summarized above, in either case wherein the coding region encodes the therapeutic peptide.

In some embodiments, administering the circular RNA to the subject includes administering to the subject exosomes that contain the circularized RNA.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Linear layout of the ceRNA construct. The identified nucleotide segments may be cloned into any desired plasmid backbone.

FIG. 2. Formation of an RNA stem loop that will create a protected triple helix structure through non-canonical base pairing with a string of polyA. The polyA is encoded directly by the sequence. (A) 5′ triple helix sequence. (B) 3′ triple helix sequence.

FIG. 3. Self-circularizing in vitro transcription. (A) Control RNA and circularized RNA+/−RNase R. (B) ceRNA with either normal NTPs (+/−RNase R) or modified with 5-methylcytidine-5′-triphosphate and pseudouridine-5′-triphosphate in equal molar ratios to the remaining canonical RNA nucleotides, ATP and GTP (+/−RNase R).

FIG. 4. ceRNA transcription induced with IPTG in E. coli. Total RNA was isolated from E. coli and then incubated with RNase R (denoted by +). RNase-R-resistant ceRNA is the sole detected RNA product after incubation with RNase R.

FIG. 5. 293T cells transfected with the DNA encoding the ceRNA (left) or transfected with ceRNA (right) express the signal protein (nano-luciferase) driven by the IRES element. Relative light units (RLU).

FIG. 6. Schematic diagram depicting transcription and circularization of ceRNA.

FIG. 7. Eukaryotic cells were plated at 100,000 cells/well the night before and transfected with DNA encoding ceRNA using Lipofectamine Stem reagent (500 ng DNA+2 μl Lipofectamine. GFP fluorescence was captured 24 hours post transfection. (A) and (B) are two independent experiments.

FIG. 8. In vitro transcribed ceRNA encoding mCherry fluorescent protein transfected into 293T cells using Lipofectamine reagent. 24 hours post transfection.

### DETAILED DESCRIPTION OF ILLUSTRATIVE EMBODIMENTS

This disclosure describes circularized polynucleotide constructs. In some cases, the circularized engineered polynucleotide is an RNA. The circularized engineered RNA (ceRNA) constructs are resistant to degradation by RNase. In other cases, the circularized polynucleotide may be a DNA that encodes an RNA with all of the components necessary for the RNA to circularize upon transcription.

The constructs described herein provide an approach to stabilizing transcribed RNA. International Patent Application No. PCT/US2017/063060 (published as International Publication No. WO 2018/098312 A2) describes certain modifications that can be made to RNA molecules to promote sustainable expression of coding regions encoded within the RNA molecule. Some of those modifications include the length of a ploy(A) tail, a 5′m7G cap, modified nucleotides, IRES modification of the 5′ UTR, and/or pseudoknot modification of the 3′UTR. This disclosure describes an alternative construct that may be engineered into an RNA molecule in place of, or in addition to, one or more the above-mentioned modifications. The constructs described herein allow one to produce RNA molecules in live cells because the RNA molecules are resistant to degradation by RNase.

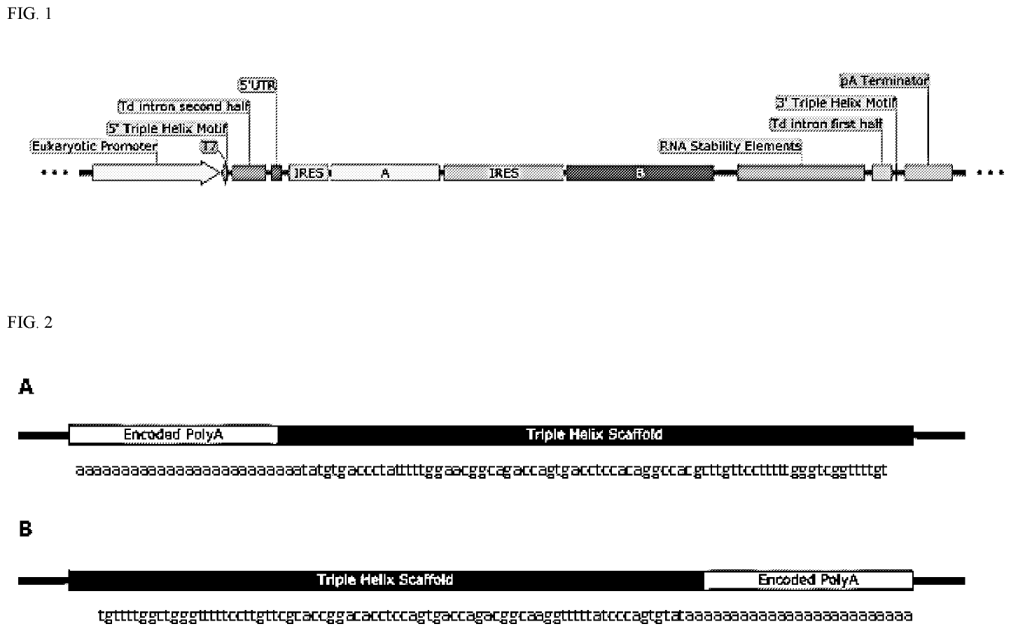

Components of the ceRNA are illustrated in FIG. 1 and include one or more promoters, a circularizing element, one or more internal ribosome entry sites (IRES), and one or more coding regions who translation is controlled by an IRES. As used herein, the ceRNA (circularized engineered RNA) terminology is used to refer to the transcribed RNA that includes all of the components listed below, whether or not the RNA is in a linear or circularized form. ceRNA can, in some uses, also refer to a polynucleotide—e.g., DNA—that encodes an RNA having all of the components listed below.

The promoter can be any suitable promoter that allows for transcription of the ceRNA in a eukaryotic cell or a prokaryotic cell. The promoter can be selected depending upon whether the ceRNA is being designed for production in a eukaryotic cell or a prokaryotic cell. In some cases, a ceRNA can include both a eukaryotic promoter and a prokaryotic promoter so that a single construct may be used in either type of cell. The promoter can be constitutive or inducible. If placed under the control of an inducible promoter, it is possible that one can control whether the ceRNA is transcribed by a cell transfected or transduced to harbor the ceRNA (either by direct transfection or as a result of being transfected to possess a DNA that encodes the ceRNA), timing of ceRNA transcription, and/or the extent of ceRNA transcription. Exemplary eukaryotic promoters include, but are not limited to, CMV, CAG, EF1α, PGK, UbC, SV40, MSCV, TRE (inducible). TEF1, GDS, GAL1, 10, CaMKIIa, CUP1, ADH1, AOX1, HSP70-rbcs2, psbA1. In transduced cells, transcription can be initiated from viral LTRs for transduced cells. Exemplary prokaryotic promoters include, but are not limited to, T7, SP6, T7lac, Endogenous RNA holoenzyme, araBAD, pL, Ptac, and trp.

The circularization element can include one or more sequences that allow the ceRNA to circularize once it has been transcribed by the cell. Generally, the circularization element includes catalytic RNA sequence elements located on opposite ends of the RNA to form appropriate catalytic sequence sites and/or motifs to allow for a self-catalyzing splice reaction of the RNA. The circularization element can be selected depending upon whether the ceRNA is being designed for expression in a eukaryotic cell or a prokaryotic cell. In some cases, a ceRNA can include both a eukaryotic circularization element and a prokaryotic circularization element so that a single construct may be used in either type of cell. An exemplary eukaryotic circularization element can include a eukaryotic splice donor at the 3′ end (when in the linear form shown in FIG. 1) and splice acceptor at the 5′ end of the linear RNA transcript. Endogenous RNA processing machinery then splices the segments to form a circular product. In some cases, one can include additional complementary sequences at each end of the linear RNA, which can bring the donor and acceptor sites into closer apposition for more efficient splicing. An exemplary prokaryotic circularization element can include an intron from thymidine synthetase (Td) of bacteriophage RNA, which allows for self-catalyzed circularization of the ceRNA construct. A first segment of the intron is places at the 3′ end of the linear ceRNA construct and a second segment of the intron is placed at the 5′ end of the linear ceRNA construct, as shown in FIG. 1. The intron segments self-catalyze circularization and, in the process of doing so, are self-cleaved.

The internal ribosome entry site (IRES) allows translation to be initiated without an open 5′ capped end. The IRES can be selected from any class of IRESes—i.e., any one of Group I-IV. In some embodiments, the IRES can be cricket paralysis virus IRES (CrPV-IRES) or Plautia stali intestine virus IRES (PSIV-IRES), each of which initiates translation in mammalian cells and requires minimal use of the cellular machinery for translation. the reduced reliance on the cellular machinery compared to other IRES sequences means that translation from CrPV-IRES and PSIV-IRES can be more efficient that when using other IRES sequences. Exemplary alternative IRES suitable for use in a ceRNA construct include, but are not limited to, IRES from hepatitis C Virus (HCV), classical swine fever virus (CSFV), foot-and-mouth disease virus (FMDV), encephalomyocarditis virus (EMCV), polio virus, or hepatitis A virus.

The coding region can include one or more polynucleotide sequences that encode a protein or therapeutic RNA whose expression is desired. FIG. 1 illustrates an exemplary construct in which a first IRES is directly upstream of coding region A and a second IRES is directly upstream of coding region B. In other embodiments, a single IRES element can initiate translation of multiple coding regions. Thus, the ceRNA can include a single IRES or multiple (e.g., two or more) IRESes. Levels of expression can be controlled by using specific IRES elements that have differing translational capabilities dependent upon cell type and cellular states. Also, when translation of multiple coding regions is under the control of a single IRES, the coding regions may be separated by a self-cleaving 2A peptide such as, for example, P2A, T2A, E2A, or F2A.

The coding region can encode any suitable protein whose expression is desired such as, for example, a therapeutic protein. For example, a subject experiencing or at risk of experiencing a major cardiac event can be treated by transforming at least some of the subject's cells with a ceRNA that encodes NAP-2, TGF-α, ErBb3, VEGF, IGF-1, FGF-2, PDGF, IL-2, CD19, CD20, and/or CD80/86 to increase the level of NAP-2, TGF-α, ErBb3, VEGF, IGF-1, FGF-2, PDGF, IL-2, CD19, CD20, and/or CD80/86 polypeptide expression. An increase in the level of one or more of these polypeptides can be used to reduce scar size and tissue remodeling and to improve cardiac function. Additional suitable therapeutic polypeptides are described in International Publication No. WO 2015/034897.

Other proteins that may be encoded by the coding region include, but are not limited to, vascular endothelial growth factor A (VEGF-A), insulin-like growth factor 1 (IGF-1), epidermal growth factor (EGF), interferon-γ, interleukin 2 (IL-2), interleukin 4 (IL-4), interleukin 10 (IL-10), avidin, streptavidin, acyloxyacyl hydrolase (AOAH), bone morphogenic protein 2 (BMP-2), bone morphogenic protein 7 (BMP-7), chondroitinase ABC (ChABC), endothelial nitric oxide synthase 3 (eNOS), peroxisome proliferator-activated receptor γ (PPARG), an antibody or fragment thereof, an antigenic peptide, an antiviral protein, or a transcription factor.

Avidin or streptavidin encoded by a ceRNA to be fused to the transmembrane segment of a protein can allow one to display the avidin protein on the outer surface of a cell. An avidin-labeled or streptavidin-labeled cell can assist with targeting biotin-labeled therapeutics such as, for example, drugs, exosomes, or immune cells.

Interleukins encoded by a ceRNA can modify the immune response and the immune cells that are recruited to target areas. For example, anti-inflammatory cytokines (e.g., IL-10 and IL-4) promote immune suppression by promoting M2 macrophage recruitment. As another example, proinflammatory cytokines (e.g., IL-8, IFNγ) can recruit immune cells (e.g., neutrophils, granulocytes) to target areas and/or activate macrophages/phagocytosis.

Acyloxyacyl hydrolase (AOAH) encoded by a ceRNA can inactivate LPS endotoxin. The AOAH may be modified to include a 5′ secretion signal and a protease-cleavable linker between the small subunit and the large subunit of AOAH. This modified form of AOAH can thereby be secreted by cells and cleaved into its active form in the circulation to catalyze the inactivation of LPS.

Bone morphogenic proteins encoded by an ceRNA can promote bone repair. For example, expressing BMP-2 and BMP-7 from the same ceRNA construct can promote forming the BMP-2/7 heterodimer for improving bone repair.

For treating spinal cord injuries, one or more proteins that mitigate the inflammatory response at the site of injury, alter the extracellular matrix, directly promote axonal growth, and/or are cytoprotective can be expressed from a ceRNA. For example, chondroitinase ABC (ChABC) and/or a modified secreted form of this protein can alter the extracellular matrix to promote axonal growth at the site of spinal cord injury.

Functional cystic fibrosis transmembrane conductance regulator (CFTR) encoded by a ceRNA can be used to treat cystic fibrosis. A ceRNA encoding CFTR can be encapsulated an inhaled in aerosolized form to introduce the ceRNA to the respiratory epithelium.

An antibody encoded by a ceRNA may be used for targeted delivery of the antibody. The antibody may be secreted by the cell harboring the ceRNA. The antibody may be a conventional full antibody, an antibody fragment, or a chimeric antibody such as, for example, a Fab, F(ab′)₂, Fab′, scFv, di-scFv, sdAb, bi-functional antibody (e.g., a BiTE or BiKE), or trifunctional antibody (e.g., TriTE or TriKE).

A ceRNA can encode an antigenic peptide so that secretion of the peptide can immunize a subject receiving the ceRNA against a pathogen that expresses the antigenic peptide.

A ceRNA can encode an anti-viral protein such as, for example, an interferon-induced transmembrane protein (IFITM) so that secretion of the anti-viral protein can limit the extent and/or severity of a viral infection.

FIG. 1 and FIG. 2 illustrate additional optional components. One may design a ceRNA to include one or more of the optional components if desired. Exemplary optional elements include, but are not limited to, a 5′ or 3′ triple helix motif, a 5′ or 3′ untranslated region (UTR), an RNA stability element, an RNA export or an RNA localization element, and/or a purification element.

A 5′ or 3′ triple helix motif is illustrated in FIG. 1 and FIG. 2. A triple helix motif creates a stem loop structure that, when associated with an encoded polyA tract, forms an RNA triple helix for protection against exonuclease activity. A triple helix motif may be from any suitable source, such as, for example, Karposi's sarcoma-associated herpesvirus (KSHV), MALAT1, Telomerase RNA pseudoknot, MENβ, or tRNA sequence.

A 5′ untranslated region (UTR) also is illustrated in FIG. 1. A UTR, whether located at the 5′ or 3′ end in the linear form shown in FIG. 1 can include, for example, an miRNA binding sequence or an extracellular vesicle targeting sequence. An miRNA binding sequence can help regulate translation from the ceRNA. The miRNA binding sequence can bind to miRNA that interferes with translation or enhances translation. Thus, by selecting an appropriate miRNA binding sequence and miRNA that binds to the miRNA binding sequence, one can provide the miRNA as desired to either “turn up” or “turn down” translation from the ceRNA. In other cases, miRNA binding can influence the overall stability of the ceRNA transcript

An extracellular vesicle targeting sequence is a nucleotide sequence that aids in shuttling the ceRNA to exosomes being released by a production cell line. For example, exosome shuttling elements utilize existing cellular machinery to shift ceRNA localization to exosomes. In this way, one can produce an exosome product that is loaded with a ceRNA molecules that encode, for example, a therapeutic protein. The ceRNA can then be delivered to a target cell as the target cell takes up the exosome. The ceRNA platform allows one to use exosome-mediated delivery of RNA that was previously unachievable. Exosomes naturally contain sufficient RNase in sufficient amounts to digest conventional forms of RNA. ceRNAs are less susceptible to RNase digestion than linear RNA constructs and, therefore, can be delivered by exosome-mediated delivery. Exemplary extracellular vesicle targeting sequences include, but are not limited to, the nucleotide sequences of SEQ ID NOs:1, 3-18.

An RNA stability element can increase stability of the ceRNA. An exemplary RNA stability element is the woodchuck hepatitis post-transcriptional regulatory element (WPRE). When transcribed, WPRE creates a tertiary structure enhancing expression. The sequence is commonly used in molecular biology to increase expression of a coding region delivered using a viral vector. When used in a 3′ UTR of an expression cassette for use in mammalian cells, WPRE can increase mRNA stability and protein yield. Other exemplary RNA stability elements include, but are not limited to, E. coli REP sequence and C-rich determinants in the 3′-UTR of globin mRNA. The stability of ceRNA allows avoidance of enzyme-driven RNA synthesis and/or allows the use of a prokaryotic or eukaryotic system to produce this RNA.

An RNA export element can increase nuclear export of the ceRNA. An RNA localization element can direct trafficking of the RNA to a particular cellular compartment.

A purification element can assist in purifying ceRNA. For example, aptamer sequences can be incorporated into the ceRNA for affinity purification of the ceRNA. As one example, SEQ ID NO:2 binds to streptavidin so that a ceRNA that contains SEQ ID NO:2 can be eluted with an excess of biotin. Other aptamer sequences designed to bind to an immobilized resin or protein may be equally suitable.

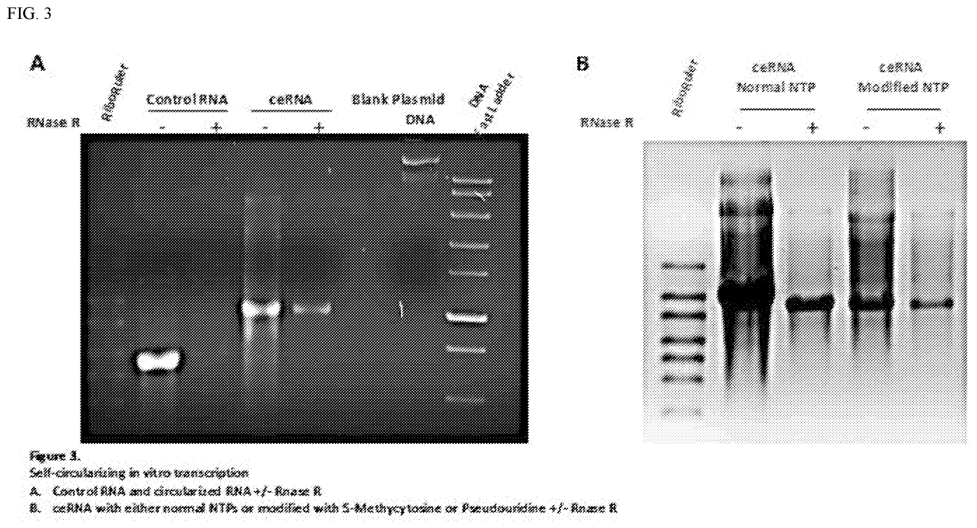

An additional modification to a ceRNA system, although not a modification to the ceRNA itself, involves stably introducing an RNase coding region into genomic DNA of the producing cell. ceRNAs produced by the cell are resistant to degradation by RNase. Modifying the producing cell to express higher levels of RNase will digest non-circularized RNA produced by the cell and, therefore, simplify purification of the ceRNA from the producing cell. FIG. 3 shows in vitro transcription of ceRNA using T7 RNA polymerase. FIG. 3A shows digestion of control linear RNA when treated with an exemplary RNase, RNase R. In contrast, ceRNA is present in the lane with ceRNA treated with RNase R. FIG. 3B shows the production of ceRNA using modified NTPs. Again, ceRNA is resistant to degradation in the presence of RNase R.

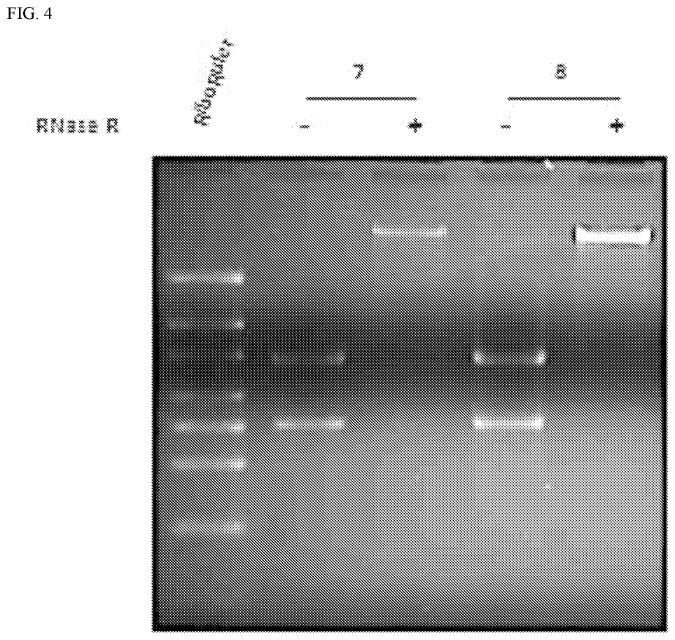

DNA plasmids containing sequences encoding ceRNA can be transformed into bacteria for transcription and subsequent circularization of the RNA via the self-splicing intron segments. The circularized RNA products can then be isolated from the total RNA of lysed cells. FIG. 4 shows transcription of ceRNA in E. coli induced with IPTG. Total RNA was isolated from the bacteria, subjected to gel purification, affinity purification using aptamer loops included in the ceRNA construct, then RNase R degradation of non-circular products. When treated with RNase R, the ceRNA is the lone RNA product detected (+ lanes). In these experiments, RNase R is used as an exemplary RNase; the ceRNAs are similarly resistant to digestion by other RNA endonucleases.

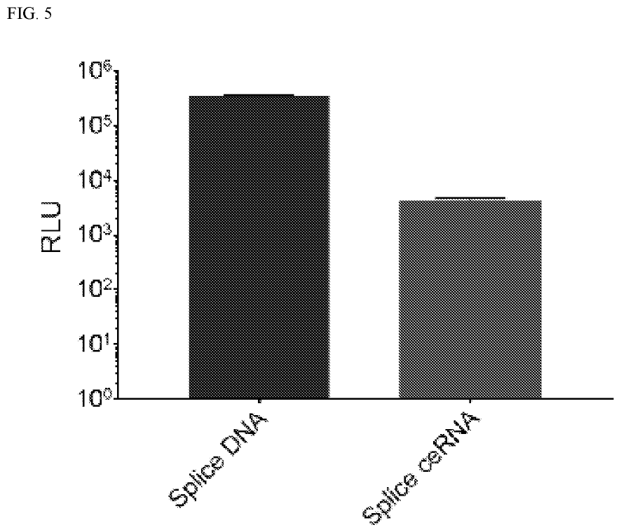

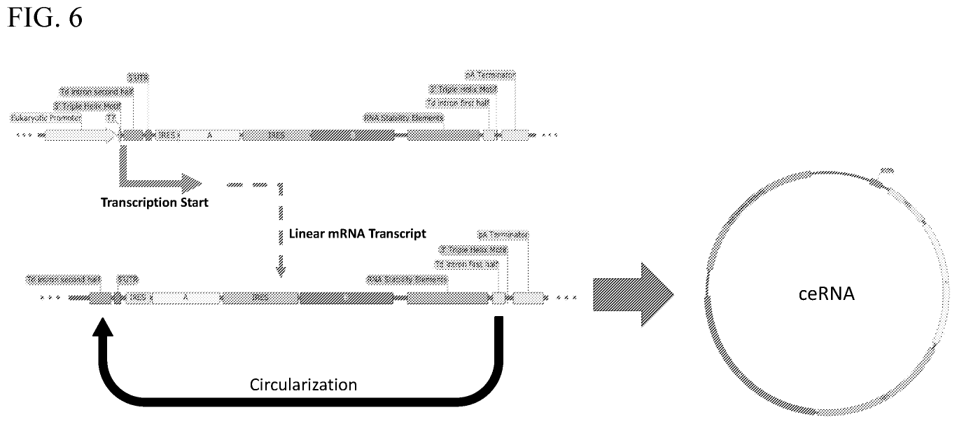

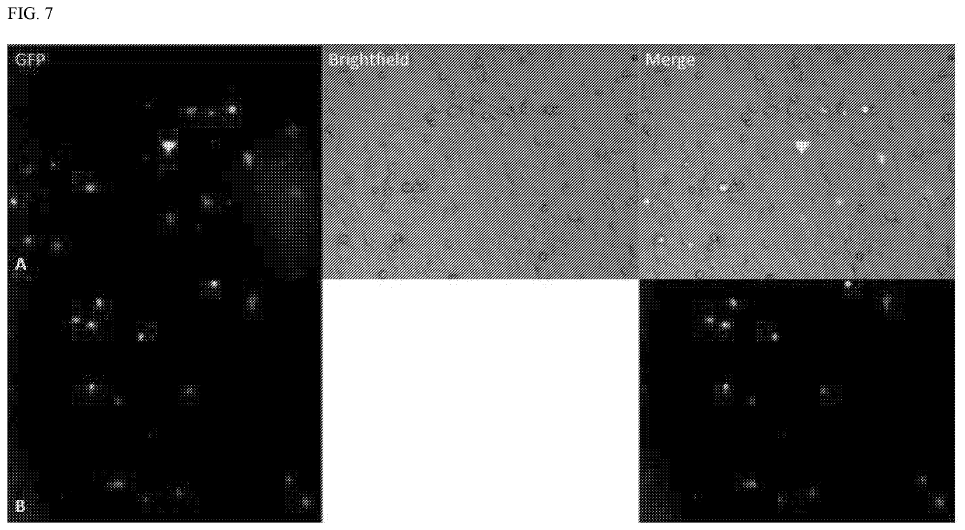

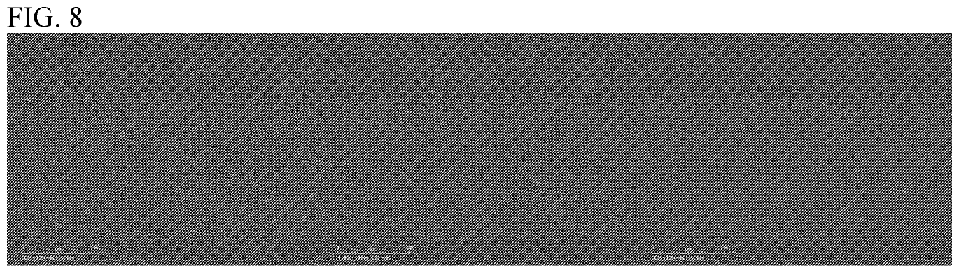

Plasmid DNA that encodes the ceRNA can alternatively be delivered to eukaryotic cells for transcription and subsequent circularization of the RNA via the self-splicing intron segments. The ceRNA plasmid sequence can be stably integrated into the host genome of the eukaryotic cell for stable production. FIG. 5 shows translation of nano-luciferase by 293T cells transfected with DNA encoding the ceRNA (left) or transfected directly with the ceRNA (e.g., Lentiviral vector in mammalian cells or recombination events in yeast).

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Using a RIBOMAX T7 in vitro transcription kit (Promega, Madison, Wis.), the reaction conditions set forth in Table 1, below, were used. Control template is provided in the kit to create a linear product of ˜1800 bases.

TABLE 1

| T7 Reaction Components | ceRNA Construct | | Control Reaction | |
|---|---|---|---|---|
| T7 Transcription 5x Buffer | 20 | μl | 4 | μl |
| rNTPs (25 mM) | 7.5 | μl Each | 1.5 | μl Each |
| DNA Template (24 ng/μl) | 40 | μl | 1 | μl + 7 μl Water |
| Enzyme Mix (T7) | 10 | μl | 2 | μl |
| Final Volume | 100 | μl | 20 | μl |

The ceRNA construct as illustrated in FIG. 1 was used as the template. The reaction was carried out at 42° C. for two hours. RNA was then isolated using an RNeasy mini kit (Qiagen, Hilden, Germany) according to manufacturer's directions. The isolated RNA was then digested with RNase R to degrade any non-circular mRNA as set forth in Table 2, below.

TABLE 2

| RNase R Reaction Components | ceRNA | Control mRNA |
|---|---|---|
| RNA (4 μg) | 5 μl | 2 μl |
| RNase R (20 U) | 1 μl | 1 μl |
| Reaction Buffer | 1 μl | 1 μl |
| Water | 3 μl | 6 μl |
| Final Volume | 10 μl | 10 μl |

The reaction was incubated at 37° C. for 20 minutes. Reaction mixtures were then mixed with 2×RNA loading buffer 1:1 and run on a 1% Agarose TBE gel+ethidium bromide.

Results are shown in FIG. 3A.

The methods are similar to described for FIG. 3A except in one reaction the RNA was modified using 5-methylcytidine-5′-triphosphate and pseudouridine-5′-triphosphate in equal molar ratios to the remaining canonical RNA nucleotides (ATP and GTP), as shown in Table 3, below.

TABLE 3

| RNase R Reaction Components | ceRNA Normal NTPs | Control mRNA |
|---|---|---|
| RNA (50 μg) | 30 μL | 30 μL |
| RNase R (100 U) | 5 μL | 5 μL |
| Reaction Buffer | 5 μL | 5 μL |
| Water | 10 μL | 10 μL |
| Final Volume | 50 μL | 50 μL |

The reaction was incubated at 37° C. for 20 minutes. Reaction mixtures were then mixed with 2×RNA loading buffer 1:1 and run on a 1% Agarose TBE gel+ethidium bromide.

Results are shown in FIG. 3B.

### Example 2

ceRNA construct was cloned into the pET 24a (+) plasmid and transformed into BL21 Star (DE3) E. coli. Cultures were grown at 30° C. until an absorbance of 0.7 at OD₆₀₀. RNA transcription was induced with 5 mM IPTG for 30 minutes, at which point 100 μg/mL of chloramphenicol was added to the lanes marked as 8 in FIG. 4. Bacterial cultures were collected 20 minutes after the chloramphenicol was added, for a total of 50 minutes of induction. RNeasy mini kit (Qiagen, Hilden, Germany) was used to extract RNA with the addition of three freeze thaw cycles to aid in cell wall and membrane rupture. Reaction were prepared as shown in Table 4, below.

TABLE 4

| RNase R Reaction Components | ceRNA | | ceRNA + Chloramphenicol | |
|---|---|---|---|---|
| RNA (1 μg) | 15 | μl | 15 | μl |
| RNase R (100 U) | 0.5 | μl | 0.5 | μl |
| Reaction Buffer | 2 | μl | 2 | μl |
| Water | 2.5 | μl | 2.5 | μl |
| Final Volume | 20 | μl | 20 | μl |

The RNA was mixed 1:1 with RNA loading buffer and run on a 1% agarose gel.

Results are shown in FIG. 4.

### Example 3

HEK 293T cells were plated on a 24-well plate at 50,000 cells/well the night prior to transfection. Either the DNA plasmid encoding for ceRNA or ceRNA directly was transfected using LIPOFECTAMINE STEM reagent (Thermo Fisher Scientific, Waltham, Mass.) according to manufacturer's directions, as set forth in Table 5, below.

TABLE 5

| Transfection Components | DNA or RNA |
|---|---|
| OPTI-MEM | 25 μl |
| LIPOFECTAMINE STEM | 2 μl |
| OPTI-MEM | 25 μl |
| DNA (500 ng) or RNA (1.5 μg) | 1 μl |

Nano-Luciferase expression is driven by the IRES element of the construct. NanoGlo assay was used to detect nLuc product in the culture wells.

Results are shown in FIG. 5.

### Example 4

Eukaryotic cells were plated at 100,000 cells/well the night before and transfected with DNA encoding ceRNA using LIPOFECTAMINE STEM reagent (Thermo Fisher Scientific, Waltham, Mass.) as set forth in Table 6, below.

TABLE 6

| Transfection Components | DNA or RNA |
|---|---|
| OPTI-MEM | 25 μl |
| LIPOFECTAMINE STEM | 2 μl |
| OPTI-MEM | 25 μl |
| DNA (500 ng) | 1 μl |

GFP fluorescence was captured 24 hours post transfection. Results are shown in FIG. 7.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

Exemplary extracellular vesicle targeting sequence
SEQ ID NO: 1
GGGACGACGATGACACGATACTTTGTCGGCCGAACTCGCTGCTCCGATCC
GGCGAGATCGCAGGGTGTTGCTATTCGCGTGCCGTGTGCATACGCCGATC
ACATGACCAGGGACGACGATGACACGATACTTTGTCGGCCGAACTCGCTG
TTTAACTGCCCGGCGAGATCGCAGGGTGTTGTGCTATTCGCGTGCCGTGT
GCATACGCCGATCACATGACCAACCCTGCCGCCTGGACTCCGCCTGT
Exemplary RNA purification element
SEQ ID NO: 2
ACCGACCAGAATCATGCAAGTGCGTAAGATAGTCGCGGGCCGGG
Exemplary extracellular vesicle targeting sequence
SEQ ID NO: 3
ACCAGGCUUGGA
Exemplary extracellular vesicle targeting sequence
SEQ ID NO: 4
UCACAUGG
Exemplary extracellular vesicle targeting sequence
SEQ ID NO: 5
CUUGGAAGCAGA
Exemplary extracellular vesicle targeting sequence
SEQ ID NO: 6
UCUUCUCGGAUU
Exemplary extracellular vesicle targeting sequence
SEQ ID NO: 7
GGCCAGGGGUUC
Exemplary extracellular vesicle targeting sequence
SEQ ID NO: 8
AGGAACGAAC
Exemplary extracellular vesicle targeting sequence
SEQ ID NO: 9
UAUGUGGCCAUC
Exemplary extracellular vesicle targeting sequence
SEQ ID NO: 10
ACCAGGCUUGGA
Exemplary extracellular vesicle targeting sequence
SEQ ID NO: 11
CAGCGAGACC
Exemplary extracellular vesicle targeting sequence
SEQ ID NO: 12
CUCACUUGGGAG
Exemplary extracellular vesicle targeting sequence
SEQ ID NO: 13
AGCACCACCU
Exemplary extracellular vesicle targeting sequence
SEQ ID NO: 14
CAGGAGUCUACA
Exemplary extracellular vesicle targeting sequence
SEQ ID NO: 15
GGAGAAGAAGGC
Exemplary extracellular vesicle targeting sequence
SEQ ID NO: 16
GGGGAACCUGCA
Exemplary extracellular vesicle targeting sequence
SEQ ID NO: 17
ACCAAUGGGG
Exemplary extracellular vesicle targeting sequence
SEQ ID NO: 18
GGGGAACCUGCA

1. A polynucleotide comprising:
a transcription unit comprising:
a circularizing element comprising a first sequence at the 5′ end of the transcription unit and a second sequence at the 3′ end of the transcription unit;
at least one coding region between first sequence of the circularizing element and the second sequence of the circularizing element; and
an internal ribosome entry site (IRES) operably linked to the coding region; and
a promoter operably linked to the transcription unit.

2. The polynucleotide of claim 1 wherein the polynucleotide comprises DNA.

3. The polynucleotide of claim 1 wherein the polynucleotide comprises RNA.

4. The polynucleotide of claim 1, wherein:
the first sequence of the circularizing element comprises a first portion of intron from thymidylate synthetase (td) of bacteriophage RNA; and
the second sequence of the circularizing element comprises a second portion of the intron from thymidylate synthetase (td) of bacteriophage RNA.

5. The polynucleotide of claim 1, wherein:
the first sequence of the circularizing element comprises a eukaryotic splice acceptor sequence; and
the second sequence of the circularizing element comprises a eukaryotic splice donor sequence.

6. The polynucleotide of claim 1, wherein the IRES comprises cricket paralysis virus IRES (CrPV-IRES) or Plautia stali intestine virus IRES (PSIV-IRES).

7. The polynucleotide of claim 1, wherein the coding region encodes a therapeutic peptide.

8. The polynucleotide of claim 1, wherein the IRES is operably linked to at least two coding regions.

9. The polynucleotide of claim 1, further comprising a second IRES operably linked to a second coding region.

10. The polynucleotide of claim 1, wherein the transcription unit further comprises:
a triple helix motif;
an untranslated region (UTR);
an RNA stability element;
an RNA export or an RNA localization element; or
an affinity purification aptamer.

11. The polynucleotide of claim 10, wherein the UTR comprises an miRNA binding site or a extracellular vesicle targeting sequence.

12. The polynucleotide of claim 10, wherein the RNA stability element comprises woodchuck hepatitis post-transcriptional regulatory element (WPRE).

13. The polynucleotide of claim 10, wherein the RNA export element comprises a sequence that promotes export of RNA from a cell nucleus.

14. A circular RNA molecule comprising:
at least one coding region; and
an internal ribosome entry site (IRES) operably linked to the coding region.

15. The circular RNA molecule of claim 14, wherein the IRES comprises cricket paralysis virus IRES (CrPV-IRES) or Plautia stali intestine virus IRES (PSIV-IRES).

16. The circular RNA molecule of claim 14, wherein the coding region encodes a therapeutic peptide.

17. The circular RNA molecule of claim 14, wherein the IRES is operably linked to at least two coding regions.

18. The circular RNA molecule of claim 14, further comprising a second IRES operably linked to a second coding region.

19. The circular RNA molecule of claim 14, further comprising:
a triple helix motif;
an untranslated region (UTR);
an RNA stability element;
an RNA export or an RNA localization element; or
an affinity purification aptamer.

20. The circular RNA molecule of claim 19, wherein the UTR comprises an miRNA binding site or a extracellular vesicle targeting sequence.

21. The circular RNA molecule of claim 19, wherein the RNA stability element comprises woodchuck hepatitis post-transcriptional regulatory element (WPRE), E. coli REP element, or a beta-globin stability element.

22. The circular RNA molecule of claim 19, wherein the RNA export element comprises a sequence that promotes export of RNA from a cell nucleus.

23. A cell transformed with the polynucleotide of claim 1.

24. A cell comprising the circular RNA of claim 14.

25. The cell of claim 24, wherein the circular RNA is synthesized outside of the cell.

26. The cell of claim 24, wherein the circular RNA is synthesized by the cell.

27. The cell of claim 26, further comprising exosomes.

28. The cell of claim 27, wherein at least a portion of the circular RNA molecules in the cell are located in the exosomes.

29. A method of making a circular RNA molecule, the method comprising:
transforming a host cell with the polynucleotide of claim 1;
allowing the host cell to transcribe a linear RNA molecule from the polynucleotide; and
allowing the circularizing element to circularize the linear RNA molecule, thereby forming a circular RNA molecule.

30. The method of claim 29, further comprising isolating at least a portion of the circular RNA molecules from the host cell.

31. The method of claim 30, further comprising:
digesting linear RNA molecules with an RNase; and
collecting undigested circular RNA molecules.

32. The method of claim 29, wherein the circular RNA molecule comprises an extracellular vesicle targeting sequence effective for transferring the circular RNA to an exosome in the host cell.

33. The method of claim 32, further comprising isolating exosomes containing the circular RNA molecules.

34. A method of treating a subject in need of therapy provided by a therapeutic peptide, the method comprising administering to the subject:
a polynucleotide comprising:
a transcription unit comprising:
a circularizing element comprising a first sequence at the 5′ end of the transcription unit and a second sequence at the 3′ end of the transcription unit;
at least one coding region between first sequence of the circularizing element and the second sequence of the circularizing element, the at least one coding region encoding the therapeutic peptide; and
an internal ribosome entry site (IRES) operably linked to the coding region; and
a promoter operably linked to the transcription unit; or
a circular RNA molecule comprising:
at least one coding region that encodes the therapeutic peptide; and
an internal ribosome entry site (IRES) operably linked to the coding region.

35. The method of claim 34, wherein administering the circular RNA to the subject comprises administering to the subject exosomes that contain the circularized RNA.
